(19) United States
(12) Patent Application Publication
(10) Pub. No.: US 2011/0288391 A1
(43) Pub. Date: Nov. 24, 2011

(54) Titanium-Based Multi-Channel Microelectrode Array for Electrophysiological Recording and Stimulation of Neural Tissue

(75) Inventors: Masaru Palakurthi Rao, Riverside, CA (US); Kevin Otto, West Lafayette, IN (US); Patrick Thomas McCarthy, West Lafayette, IN (US)

(73) Assignee: PURDUE RESEARCH FOUNDATION, West Lafayette, IN (US)

(21) Appl. No.: 12/875,013

(22) Filed: Sep. 2, 2010

Related U.S. Application Data
(60) Provisional application No. 61/346,220, filed on May 19, 2010.

(51) Int. Cl. A61B 5/04

(52) U.S. Cl. 600/373

(57) ABSTRACT

A microelectrode array including a top portion, a plurality of pads positioned on the top portion, and a shank portion, the shank portion including a titanium substrate, a dielectric structure positioned on the titanium substrate, and a metallization layer embedded in the dielectric structure, the metallization layer including a plurality of electrode sites distributed longitudinally along the shank portion, and a plurality of electrical traces, wherein the dielectric structure provides an access window over each of the plurality of the electrode sites and each of the plurality of electrical traces electrically connects a corresponding electrode site of the plurality of electrode sites to a corresponding pad of the plurality of pads.

### CROSS-REFERENCE TO RELATED APPLICATIONS

This application claims priority from a U.S. Provisional Patent Application No. 61/346,220, filed on May 19, 2010, the contents of which are incorporated herein by reference in its entirety.

### TECHNICAL FIELD

The present invention generally relates to electrodes for acquiring electrical signals from physiological systems as well as providing electrical stimulation, and particularly to microelectrodes for neural applications.

### BACKGROUND

The fields of neural stimulation with electrical signals and recording neural activity by recording and analyzing electrical signals have improved our understanding of neurophysiology. These fields have also provided significant opportunities for restoring neurological functions lost to disease, stroke, or injury, as exemplified by neural prostheses that have enabled restoration of rudimentary auditory perception and control of assistive instrumentation for those with motor dysfunction, as reported in prior art. Typically microelectrodes are utilized for penetration into various areas of a subject's brain under study or treatment. However, significant challenges remain with the microelectrodes of the prior art.

One of the primary challenges is related to reliability of the microelectrodes. The microelectrodes of prior art commonly rely on silicon as a substrate material which defines the microelectrode's structural characteristics. Unfortunately, silicon is an intrinsically brittle material. This brittleness, which arises from the low fracture toughness of silicon, provides a predisposition for failure by fracture. The propensity for fracture adversely affects reliability, since fracture often results in complete loss of device functionality.

In addition, various neural applications may require various length microelectrodes. Neural applications can include stimulation and recording of the cortex and/or deep structures, such as the thalamus. Cortical recording and stimulation can be accomplished with shorter microelectrodes (about 2 mm for a rat model), while thalamic recording and stimulation requires longer electrodes (4.9 and 5.4 mm). Notably, longer silicon based microelectrodes, needed for deep brain probing and stimulation, are further susceptible to fracture.

Failure of the silicon-based microelectrode by fracture, in addition to typically rendering the device inoperable for its intended purposes, may result in fragmentation of the microelectrode within the brain. This failure mode may result in further short term and long term complications for the subject.

While, silicon-based microelectrodes can be made with more robust (i.e. increase ultimate load bearing capability) by increasing the cross sectional areas of the device, a high aspect ratio (i.e., large length with respect to small cross sectional area) of the microelectrode is desirable. The small cross sectional area minimizes tissue trauma. Typically, maximum cross sectional areas of microelectrodes are dictated by the type of applications in which the electrodes are used. For example, cross sectional area of a microelectrode used to penetrate a subject's brain may need to be small. Due to the relationship between length and cross sectional area (a smaller cross sectional microelectrode requires a smaller length to avoid failure modes, e.g., fragmentation of the microelectrodes due to buckling-induced fracture), the cross sectional area places a practical length limitation on the microelectrode. This limitation reduces access to sub-cortical structures and largely precludes extension towards simultaneous recording within precisely-defined cortical and sub-cortical regions. The latter capability is of particular interest, since it may enhance understanding of important neural processing networks, such as the corticothalamic loops that underlie auditory, visual, and somatosensory processing, as reported in the prior art.

While a robust microelectrode is highly resistant to fracture under loads associated with penetration into a subject's brain, the robust microelectrode also has to provide a suitable signal to noise ratio for recording of electrical signals. A polycrystalline diamond-based microelectrode, may be highly flexible, and may be made with a thin cross sectional area with sufficient stiffness for cortical penetration, as reported in the prior art. However, low signal to noise ratio observed during neural recording may be a limiting factor for this type of microelectrode in neural applications.

Researchers have explored other alternative materials for construction of microelectrodes. An example of an alternative material to silicon is ceramics, as reported in the prior art. A microelectrode constructed with ceramics offers only limited benefit as compared to silicon, with regard to reliability. The benefit is limited because ceramic-based microelectrodes have similar or greater propensity for fracture, as reported in the prior art.

Another group of material reported in the prior art used for manufacturing microelectrodes is polymers. A polymer-based microelectrode may possess sufficient toughness to mitigate fracture. However, due to the low modulus of elasticity associated with polymers, a polymer-based microelectrode typically requires a trade-off between device stiffness and functional reliability (i.e., ability to reliably insert the microelectrode into physiological tissue). One such trade-off is between relatively large cross sectional areas, which are required to ensure insertion reliability and recording site placement accuracy, as reported in the prior art, as well as an increase of tissue damage due to the larger cross sectional areas.

A robust microelectrode also requires biocompatibility for acute and long term uses of the microelectrode. Reported metal-based microelectrodes provide certain advantages due to high fracture toughness, which can result in plastic deformation (i.e., permanent deformation after unloading) as compared to fracture when the microelectrode is subjected to a load. Moreover, due to the high modulus of elasticity associated with metals, a metal-based microelectrode could be made with a small cross sectional area. However, despite these advantages, metal-based microelectrodes found in the prior art have limited applicability in neural applications. For example, in one prior art microelectrode, gold coating was applied to an underlying nickel structural to prevent exposure to the physiological environment. However, there are concerns about potential for release of cytotoxic nickel ions in the event of coating failure.

In addition, the microelectrodes of the prior art are limited to single-subsystem measurement or stimulation. In many cases, it is advantageous to stimulate one neural subsystem and measure electrical signals in another neural subsystem in order to study interactions between these subsystems. Currently, these studies are performed by inserting a wire microelectrode to provide the electrical stimulation with another electrode inserted for measuring electrical signals. This constrains the ability to sample and stimulate a large numbers of discrete neurons or neuronal ensembles, since increasing numbers of wire microelectrodes are required, which increases tissue trauma and logistical challenge.

Therefore, there is a need for a microelectrode array that can be designed with a high aspect ratio, with a material that provides a relatively high modulus of elasticity, with fragmentation-failure resistance, with non-cytotoxicity, with insertion reliability, and with a high signal-to-noise ratio of electrophysiological recordings.

### SUMMARY

A microelectrode array has been developed.

In one form thereof, the microelectrode array includes a top portion, a plurality of pads positioned on the top portion, and a shank portion. The shank portion includes a titanium substrate, a dielectric structure positioned on the titanium substrate, and a metallization layer embedded in the dielectric structure. The metallization layer includes a plurality of electrode sites distributed longitudinally along the shank portion, and a plurality of electrical traces. The dielectric structure provides an access window over each of the plurality of the electrode sites. Each of the plurality of electrical traces electrically connects a corresponding electrode site of the plurality of electrode sites to a corresponding pad of the plurality of pads.

In another form thereof, the microelectrode array includes a top portion, a plurality of pads positioned on the top portion, and a shank portion. The shank portion includes a titanium substrate, a dielectric structure positioned on the titanium substrate, and a metallization layer embedded in the dielectric structure. The metallization layer defines a first plurality of electrode sites and a first plurality of electrical traces positioned on a first shank portion. Each electrical trace of the first plurality of electrical traces connects a corresponding electrode site of the first plurality of electrode sites to a corresponding pad of the plurality of pads. The metallization layer also defines a second plurality of electrode sites and a second plurality of electrical traces positioned on a second shank portion. Each electrical trace of the second plurality of electrical traces connects a corresponding electrode site of the second plurality of electrode sites to a corresponding pad of the plurality of pads. Each electrode site of the first and second pluralities of electrode sites is substantially free of the dielectric structure, where the first and second pluralities of electrode sites are cleaned by an electrolysis process to remove remnants of the dielectric structure.

### BRIEF DESCRIPTION OF DRAWINGS

FIG. 1 is a front view schematic of a first embodiment of a microelectrode array with a bottom portion magnified to depict various features of the array;

FIG. 2 is a front view schematic of a second embodiment of a microelectrode array with a midsection and a bottom section magnified to depict various features;

FIGS. 3A and 3B are scanning electron micrographs of the microelectrode array of FIG. 1 at varying magnifications;

FIGS. 4A, 4B, 4C, 4D, 4E, 4F, 4G, and 4H depict various process steps included in the manufacturing of the microelectrode arrays of FIGS. 1 and 2;

FIG. 5 is a graph of experimentally measured buckling loads for microelectrode arrays with various designs of the arrays of FIGS. 1 and 2 vs. critical buckling loads for fixed-free and fixed-pinned end conditions;

FIGS. 6A and 6B are graphs of results of electrochemical impedance spectroscopy (FIG. 6A) and cyclic voltammetry (FIG. 6B) testing of microelectrode arrays of FIGS. 1 and 2 in various states;

FIGS. 7A and 7B are scanning electron micrographs illustrating permanent deformation of the microelectrode arrays of FIGS. 1 and 2 after unloading;

FIGS. 8A and 8B are graphs of results of electrochemical impedance spectroscopy (similar to FIG. 6A) and cyclic voltammetry (similar to FIG. 6B) testing of microelectrode arrays of FIGS. 1 and 2 before and after a cleaning process;

FIGS. 9A and 9B are optical micrographs of reflectivity of electrode site before and after the cleaning process; and

FIG. 10 is a graph of extracellular recordings (voltage vs. time) from auditory cortex (top 8 traces) and from auditory thalamus (bottom 8 traces) of an anesthetized rat for 16 channels of the microelectrode arrays of FIG. 2.

### DETAILED DESCRIPTION

For the purposes of promoting an understanding of the principles of the invention, reference will now be made to the embodiments illustrated in the drawings and described in the following written specification. It is understood that no limitation to the scope of the invention is thereby intended. It is further understood that the present invention includes any alterations and modifications to the illustrated embodiments and includes further applications of the principles of the invention as would normally occur to one of ordinary skill in the art to which this invention pertains.

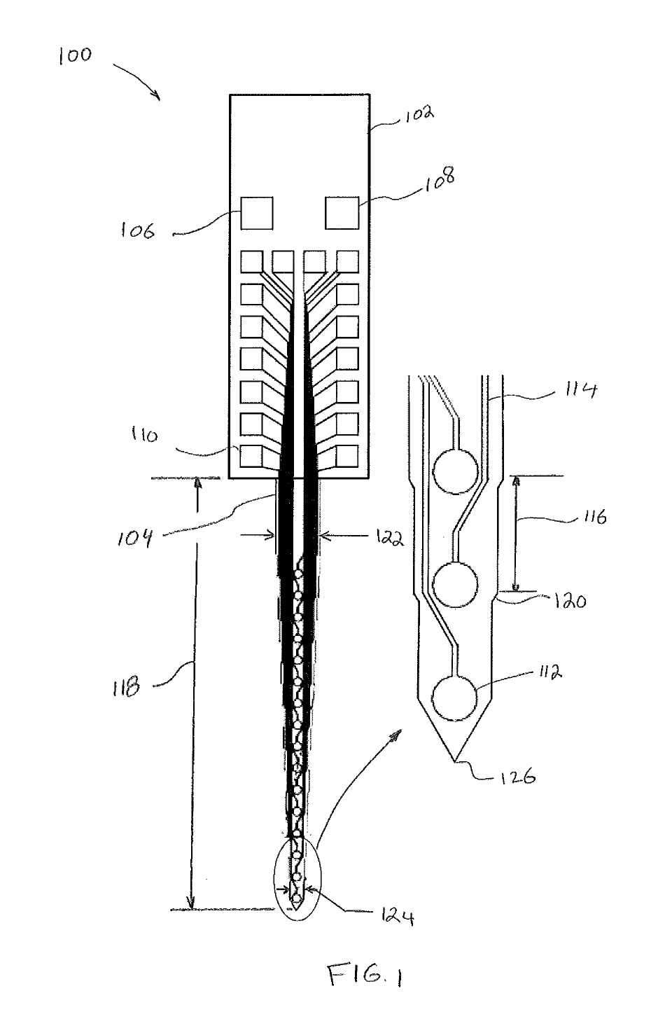

Referring to FIG. 1, a front view schematic of a first embodiment of a microelectrode array 100 is provided. A bottom portion of the array 100 is magnified to depict various features of the array. The array can be used for single subsystem (e.g. cerebral cortex) neural applications for short-term and with minor modifications for long-term recording and stimulation of neural subsystems. Exemplary neural subsystems targeted for these microelectrode arrays include auditory cortex and auditory thalamus, which are sequential processing stations in the auditory system. It is well established that the auditory cortex receives sensory input from the auditory thalamus. While the input is predominantly relayed from the thalamus to the cortex, the corticothalamic loop is a complex circuit that includes ascending, descending, and recurrent sets of neuronal connections, as discussed in the prior art. Therefore, the information exchange is not only from thalamus to cortex, but also from cortex to thalamus. Study of the cortex cannot be properly performed without taking into account the entire corticothalamic loop, as reported in the prior art. While the neural subsystems referenced in the present disclosure are located within a subject's brain, the reader should appreciate the devices of the present disclosure are applicable to neural subsystems distributed throughout a subject's body.

### Features

The array 100 comprises a T-shaped structure which includes a top portion 102 connected to a shank portion 104. The top portion 102 includes a plurality of pads 106, 108, and 110. Two pads 106 and 108 are provided to verify the quality of the electrical insulation of the dielectric layers. These pads 106 and 108 may also be used to ground the array 100 by connecting these pads (106 and 108) to electrical ground of a printed circuit board. Sixteen (16) pads 110 positioned on the top portion 102 selectively connect to sixteen (16) electrode sites 112 distributed longitudinally along the shank portion 104.

Each electrode site 112 is connected to the associated pad 110 via an electrical trace 114. The electrode sites 112 are separated by a pitch 116 along a length 118 of the shank portion 104. With every additional electrode site 112, the shank portion 104 widens by a chamfer 120 such that the shank portion 104 begins with a maximum shank width 122 and ends with a minimum shank width 124. The shank portion finally terminates at a tip 126.

Exemplary dimensions for the array 100 include 50, 75, and 100 μm for the pitch 116, 5 μm for width of each electrical trace 114, and 40 μm or 23 μm diameters for electrode sites 112. In arrays with 23 μm diameter electrode sites 112, the shank portion is defined by a width that is tapered from a minimum width 124 of about 48 μm to a maximum width 122 of about 192 μm where the shank portion 104 connects to the top portion 102. In the arrays with 40 μm diameter electrode sites 112, shank width is ranged from 65-209 μm.

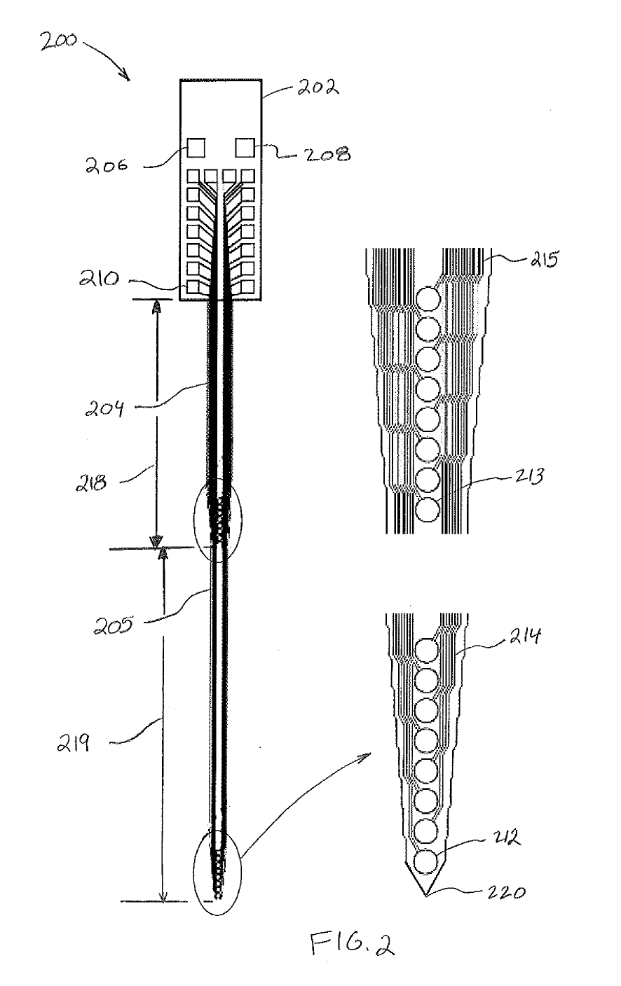

Referring to FIG. 2, a front view schematic of a second embodiment of a microelectrode array 200 is provided. Bottom portion and a midsection portion of the array 200 are magnified to depict various features of the array. The array 200 comprises a T-shaped structure and includes a top portion 202, and two shank portions 204 and 205. The top portion 202 includes verification/ground pads 206 and 208, and a plurality of pads 210. The two shank portions 204 and 205 include two pluralities of electrode sites 212 and 213, and two associated pluralities of electrical traces 214 and 215 which selectively connect each of the two pluralities of electrode sites 212 and 213 to a respective pad of the plurality of pads. The two shank portions 204 and 205 are defined by lengths 218 and 219. Exemplary dimensions of the array 200 include 2 mm for the length 218 and 2.9 mm and 3.4 mm for the length 219.

While the array 100 is suitable for stimulating and recording electrical signals from a single neural region (i.e., within a single neural subsystem), the array 200 is suitable for stimulating and recording activity from two regions. For example, the dual-region microelectrode array 200 developed according to the present disclosure enables simultaneous recording and stimulation of cortical and thalamus subsystems. Therefore, the interactions between the two neural subsystems can be studied without the need for inserting two different electrodes. Furthermore, the provision for multiple electrode sites (i.e., 112 in FIGS. 1 and 212 and 213 in FIG. 2) provide a multi-sampling capability which improves signal reliability, especially in cases where one or multiple of the electrode sites become inoperable in acute or long-term procedures. Both in vitro and in vivo uses of the microelectrodes 100 and 200 are further described below.

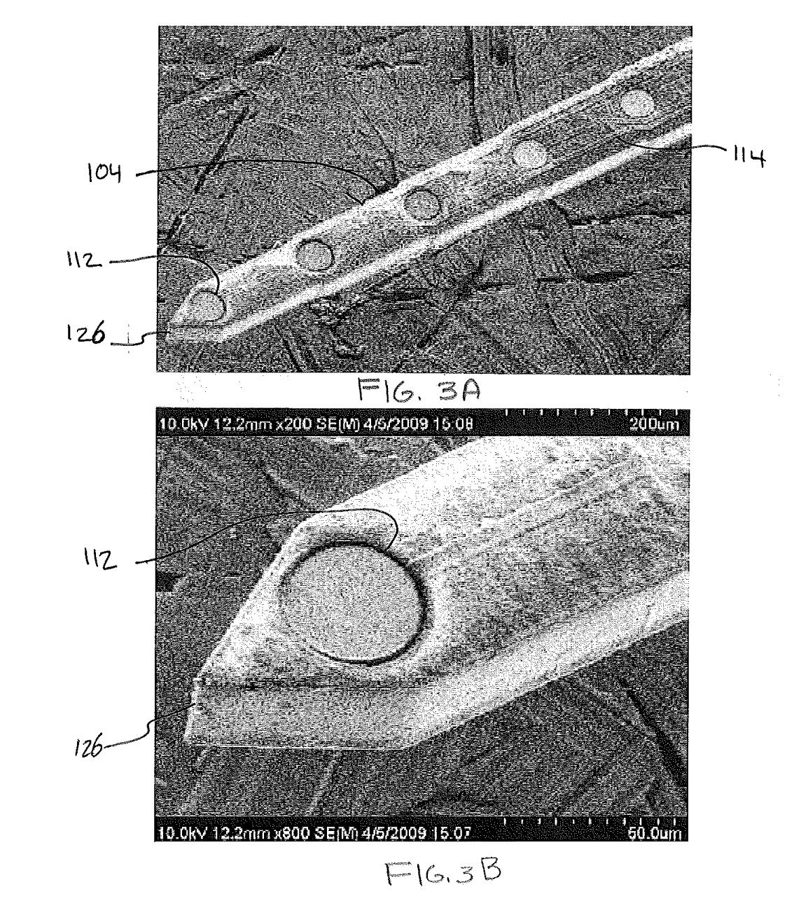

Referring to FIGS. 3A and 3B, scanning electron micrographs of portions of microelectrode arrays 100 and 200 are depicted at two different magnifications (references associated with array 100 are provided, however, FIGS. 3A and 3B are applicable to both arrays 100 and 200). As depicted in these figures the shank portion 104 is defined by substantially rectangular cross sections which terminate at a wedge-like point 126.

### Fabrication

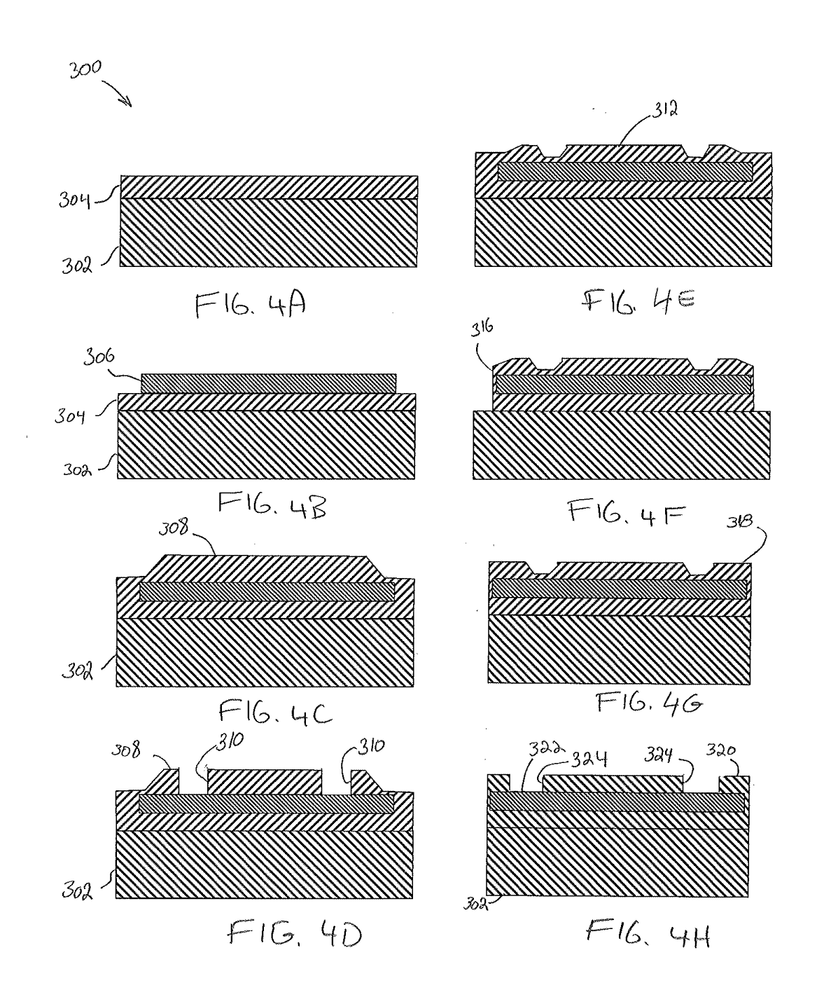

Referring to FIGS. 4A, 4B, 4C, 4D, 4E, 4F, 4G, and 4H process steps 300 for manufacturing the microelectrode arrays depicted in FIGS. 1 and 2 are provided. A titanium foil 302 is used as a substrate. The titanium substrate can be obtained from Fine Metals Corp, Ashland, Va. (e.g., Gr 1 Ti, 99.7% Ti,). An exemplary thickness of the titanium (Ti) foil 302 is 25.4±7.62 μm. The titanium foil was first cleaned by ultrasonic agitation in acetone and isopropanol, respectively, followed by deionized (DI) water rinsing and nitrogen drying.

Referring to FIG. 4A, a 0.6 μm layer of silicon oxide (SiO₂) dielectric 304 is deposited by plasma-enhanced chemical vapor deposition (PECVD) to insulate the subsequently placed electrical structures from the substrate. Example of the PECVD process is performed by Benchmark 800 CVD from Axic Inc, Santa Clara, Calif. Exemplary process conditions for SiO₂ include pressure at 230 mT (militorr), radiofrequency (RF) power at 26 W, 200 standard cubic centimeter per minute (sccm) nitrous oxide (N₂O), 35 sccm 5% silane (SiH₄), and temp at 300° Celsius (C). The substrate 302 is cleaned by a suitable solvent known in the art, and thereafter mounted to a 100 mm silicon (Si) carrier wafer with a thermally conductive adhesive tape (9882, 3M Electronics, St. Paul, Minn.).

Referring to FIG. 4B, a metal layer 306 for electrode sites (112, 212, and 213), electrical traces (114, 214, and 215), and pads (110, and 210, as well as verification/ground pads) is deposited using an electron beam deposition process and are patterned with known photolithographic liftoff techniques. The metal layer 306 includes 20 nm Ti and 500 nm gold (Au) and is deposited using an electron beam deposition system from CHA Industries, Fremont, Calif. (e.g., CHA SE-600). Process conditions for deposition of Ti using the electron beam deposition process include 1.0×10⁻⁶ torr (T), at a rate of 1 Å/s. Process conditions for deposition of Au using the electron beam deposition process include 1.0×10⁻⁶ T, at a rate of Å/s. The metal layer 306 is deposited on the first dielectric layer 304. Following the metal deposition step (FIG. 4B), the assembly is soaked in acetone to release the Ti foil from the Si carrier wafer and then cleaned with solvents known in the art.

The electron beam deposition process is one exemplary process that can be used for depositing metal. Other processes include other chemical vapor deposition (CVD)-based techniques, electroplating, or metal sputtering operation. Furthermore, a number of additional coating materials for the electrode sites specifically are known in the art, including titanium nitride, iridium oxide, Polyethylenedioxythiophene (PEDOT), carbon nanotubes, etc. Each of these materials is intended to improve charge transfer capacity and is also compatible with the Ti microelectrode fabrications processes described herein.

Referring to FIG. 4C, a second dielectric layer 308 is deposited by PECVD. The second dielectric layer includes 0.2 μm silicon nitride (Si₃N₄) and 0.8 μm of SiO₂, which are deposited in sequential order by a PECVD. The second dielectric layer 308 is provided to insulate the assemblies depicted in FIGS. 4A and 4B from the surrounding environment. Process conditions for depositing Si₃N₄ include pressure at 400 mT, RF Power at 100 W, 100 sccm NH₃, 120 sccm 5% SiHₜ, and Temp at 300° C. The dual layer stack Si₃N₄/SiO₂ promotes adhesion to the underlying Au layer and minimize stress-induced curvature of the devices arising from intrinsic stresses and thermal expansion mismatch between the deposited film and the underlying Ti substrate.

After depositing the second dielectric layer 308, the assembly depicted in FIG. 4C is again mounted to a 100 mm Si carrier wafer with a thermally conductive tape. Referring to FIG. 4D, windows 310 for the electrode sites (112, 212, and 213) and the pads (110 and 210) are opened via a photolithographic patterning and dry etching process of the second dielectric layer 308 by an E620 R&D made by Panasonic Factory Solutions, Japan. Exemplary process conditions include pressure at 1.00 Pascal (Pa), RF source forward power at 500 W, RF bias forward power at 400 W, and 40.0 sccm trifluoromethane (CHF₃). The photoresist layer used in the etching process is stripped and the assembly depicted in FIG. 4D is soaked in acetone to release the Ti foils from the Si wafer carrier.

Referring to FIG. 4E, a third dielectric layer 312 including 0.2 μm Si₃N₄ and 3.00 μm SiO₂, is then deposited by PECVD to provide an etch mask for the subsequent deep etch of the underlying Ti substrate. The assembly depicted in FIG. 4E is re-mounted to a Si carrier wafer, and profiles of the shank portions (104, 204, and 205) are patterned and transferred into the dielectric layers via dry etching as depicted in FIG. 4F, forming the dielectric layer 316. Process conditions for the etching process include pressure at 0.25 Pa, RF source forward power at 900 W, RF bias forward power at 200 W, and 40.0 sccm CHF₃. The photoresist in the etching process is then stripped.

Profiles for the shank portion (104, 204, and 205) are transferred through the underlying Ti substrate using a titanium inductively coupled plasma deep etch (TIDE) process performed by the E620 R&D, as depicted in FIG. 4G, resulting in a dielectric layer 318. The process conditions include pressure at 2.0 Pa, RF source forward power at 400 W, RF bias forward power at 100 W, 100 sccm of chlorine (Cl₂), and 5 sccm of Argon. The assembly depicted in FIG. 4G is subjected to a final short dry etch to remove the remaining thin dielectric layer protecting the electrode sites and pads, followed by a soaking process in acetone to release from the Si carrier wafer, as depicted in FIG. 4H. The result include a dielectric structure 320 formed on the titanium substrate 302 with a patterned metal layer 322 is embedded within the dielectric structure 320. Windows 324 formed on the patterned metal layer 322 provide electrical access to the metal layer 322. The windows 324 are formed over the electrode sites (112, 212, and 213), as depicted in FIGS. 1 and 2. Revisiting FIGS. 3A and 3B, the smooth vertical sidewalls resulting from the TIDE process are clearly depicted, as is the integrity of the dielectric and metal layers deposited on the Ti substrate.

### In Vitro Testing

The microelectrode arrays 100 and 200 were tested in vitro prior to being used in vivo. The in vitro testing was for at least two reasons. One goal for the in vitro studies was to determine adequacy of Ti-based arrays based on a comparison with available Si-based arrays prior to undertaking in vivo studies. Another goal of the in vitro characterization studies was to determine whether Ti-based arrays were able to maintain recording functionality after being subjected to buckling-induced elastic and/or plastic deformation.

### Mechanical Characteristics

Since the buckling characteristics of the microelectrode arrays depicted in FIGS. 1 and 2 is one of the important attributes of these arrays, the fabricated arrays were tested for buckling. The buckling behavior of the microelectrode arrays can be assessed by longitudinal uniaxial compression testing. Both the Ti-based microelectrode arrays based on the present disclosure and commercially available silicon microelectrode arrays from NeuroNexus Technologies, Ann Arbor, Mich. (e.g., A1×16−5 mm, 100-413, which is designation for a cable-less probe, one shank with sixteen electrode sites per shank, with 5 mm long shank and 100 μm site spacing with 413 μm² site area) were tested for buckling. The silicon-microelectrodes were tested for performance benchmarking purposes.

The arrays were mounted to a silicon carrier chip using either a cyanoacrylate adhesive (Pacer Technology, Rancho Cucamonga, Calif.) or a double-sided carbon tape. The mounted devices were then attached to a manually-driven micromanipulator (M3301R, World Precision Instruments Inc., Sarasota, Fla.), which was utilized to load the device tips against a microbalance scale (AB54-S/FACT, Mettler Toledo, Columbus, Ohio). Forces exerted by the tips of the arrays during testing were recorded using the microbalance and the arrays were carefully observed for buckling and fracture via a charge coupled device (CCD) with magnifying optics. Five microelectrodes were tested for each length variations (i.e. 2 mm, 4.9 mm, and 5.4 mm).

The average critical buckling forces of the Ti-based microelectrode arrays were 99.80±20.70 mN, 20.88±4.18, and 19.41±4.41 for the 2.0 mm, 4.9 mm, and 5.4 mm length arrays, respectively. In contrast, the average measured elastic buckling load for five 5 mm shank length commercially-available silicon arrays was observed to be 3.1±0.65 mN.

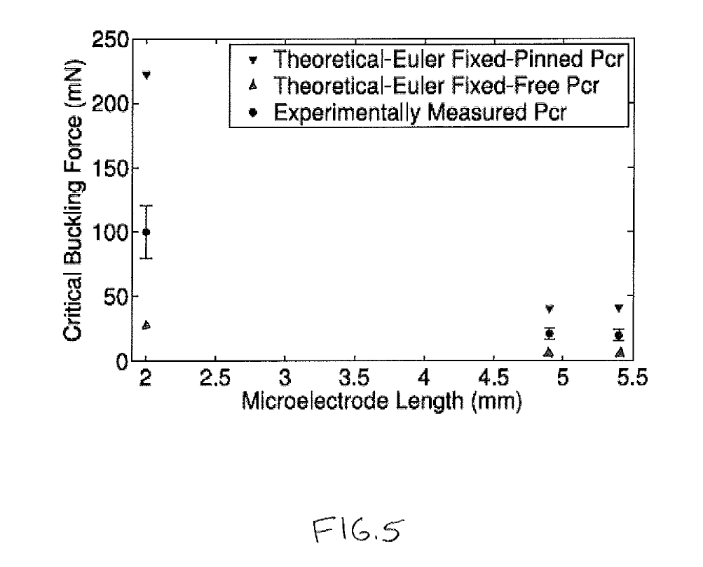

The above measured elastic buckling loads can also be contrasted to theoretical critical buckling loads, P_{cr}, estimated using Eulerian buckling analysis. In the theoretical analysis the microelectrode arrays can be modeled as long and slender columns under uniaxial longitudinal compressive loading. The P_{cr} is governed by

$$\begin{matrix}{P}_{cr} = \frac{{π}^{2} EI}{{L}_{e}^{2}} & ( 1 ) \\ I = \frac{{wt}^{3}}{12} & ( 2 )\end{matrix}$$

where E is the modulus of elasticity (Eₛᵢ=166 GPa, E_{Ti}=107 GPa, as reported in the prior art),
I is the moment of inertia,
Lₑ is the effective shank length,
w is the shank width, and
t is the shank thickness. The effective shank length is governed by the choice of end support conditions, with previous studies demonstrating fixed-free or fixed-pinned conditions as the most appropriate for the given experimental conditions. Critical buckling load estimations were performed for each device length with Lₑ=2L (fixed-free) and Lₑ=0.7L (fixed-pinned), where L was taken to be the actual device shank length (i.e., 2 mm, 4.9 mm, or 5.4 mm) Referring to FIG. 5, a graph of experimentally measured buckling loads for microelectrode arrays with various designs of the arrays of FIGS. 1 and 2 vs. theoretical Euler critical buckling loads for fixed-free and fixed-pinned end conditions is provided.

To simplify calculation of the theoretical critical buckling loads reported in FIG. 5, the shank portions (104, 204, and 205) were approximated as uniform columns (i.e., columns with constant modulus of elasticity, thickness, and width). The effective width of the shank portion can be defined as the width necessary to maintain equivalent planar shank area for the length of the shank portion. The shank thickness was defined as the sum of the thicknesses of the Ti foil used for the device fabrication (about 25 μm+/−10 μm) and the dielectric layer stack (about 1.5 μm). Scanning electron microscope measurements performed on selected microelectrode arrays of FIGS. 1 and 2 indicated actual thicknesses ranging from 34.2-37.6 μm with an average thickness of 35.2 μm for the Ti substrate and the dielectric stack. The modulus of elasticity of the column material was taken to be that of titanium, since the contribution of the dielectric layer stack is minimal as compared to the Ti substrate, and further due to the relatively small mismatch between the moduli of elasticity of the two major constituents attached to the Ti (i.e., SiO₂, where E_{SiO2}=70 GPa and Au, where E_{Au}=80 GPa, as reported in the prior art).

Finite element analyses performed for selected design variants with actual device dimensions demonstrated excellent agreement with analytical solutions, thus suggesting that the underlying simplifying assumptions used for the analytical solutions did not introduce significant error.

Subsequent in vivo studies, demonstrated that the Ti-based microelectrode arrays possessed sufficient stiffness to penetrate both rat pia and dura, unlike comparable silicon-based devices, which typically require retraction of the dura matter prior to insertion, as reported in the prior art. While the experimental critical buckling loads measurements suggest that the tested Si-based arrays also possess sufficient stiffness for cortical insertion (through pia, but not dura), margin of reliability is significantly reduced relative to the Ti devices (buckling load range=1.85 mN to 4.10 mN).

A comparison of Si and Ti-based arrays with equivalent dimensions (i.e. w, t, and Lₑ, see equations 1 and 2 above) is governed by the following equation:

$${P}_{Ti} = \frac{{E}_{Ti}}{{E}_{Si}} {P}_{Si} \qquad ( 3 )$$

As provided in the equation 3 above, the critical buckling loads for Si and Ti-based arrays can be associated through the ratio of the respective elastic moduli. Substitution of appropriate moduli values into Eq. 3 reveals that the predicted critical buckling load for a Ti microelectrode array would be approximately 66% of a comparable Si device.
While, the lower buckling load for Ti-based microelectrodes may seem to reduce performance of the Ti-based arrays, there is a practical limitation for the applications in which the Si arrays can be used. Specifically, the intrinsic brittleness of silicon limits its plasticity, thus resulting in onset of fracture soon after the elastic buckling limit has been exceeded. In contrast, the Ti-based arrays eliminate the aforementioned limitation. In particular, in a Ti-based array after the initial elastic buckling limit is reached, the shank portion (104, 204, and 205, as depicted in FIGS. 1 and 2) continue to plastically deform without fragmentation or fracture. In addition, the ability of Ti-based arrays to plastically deform indicates potential for additional load bearing capacity beyond the onset of elastic buckling As such, Ti-based microelectrode arrays provide greater tolerance to overloading during insertion and largely preclude potential for catastrophic fragmentation within the brain, thus increasing reliability relative to conventional silicon devices.

### Electrical Characteristics

The electrical performance of the microelectrode arrays of FIGS. 1 and 2 and the effect of plastic deformation on recording functionality were assessed in vitro using electrochemical impedance spectroscopy (EIS) and cyclic voltammetry (CV). Since silicon-based arrays are expected to fail catastrophically and, therefore, lose recording functionality, in these in vitro studies only Ti-based arrays were tested.

The microelectrode devices were packaged by bonding to commercially-available printed circuit boards (PCBs) (A-16, NeuroNexus) using a cyanoacrylate adhesive. Gold wire-bonding was used to make connections between the pads on the arrays and their respective bond pads on the PCBs (7400A, West-Bond, Anaheim, Calif.). An additional layer of cyanoacrylate is then applied over the contact pad area as an encapsulant to protect the exposed wires.

These packaged devices were electrically tested using a three-electrode test apparatus, which included a manually-operated force applicator connected to an electrode array immersed in a 1× phosphate buffered saline (PBS) solution at room temperature inside a glass beaker. The test setup also included a calomel electrode (Fisher Scientific, Waltham, Mass.) used as a reference electrode with a platinum wire serving as a counter electrode. The microelectrode-bearing PCB was connected to a wiring harness attached to ribbon cabling leading to an Autolab potentiostat PGSTAT12 (EcoChemie, Utrecht, The Netherlands) with built-in frequency analyzer (Brinkmann, Westbury, N.Y.). The wiring harness was also affixed to the manually-operated force applicator which allowed variation of the distance between the microelectrode tip and the bottom of the glass beaker. The test apparatus was isolated within a copper mesh “Faraday” cage. A 25 mV root mean square (RMS) sine wave was applied to electrode sites for EIS tests with frequencies ranging logarithmically from 0.1 to 10 kHz. CV testing was performed using a linear voltage sweep from −0.6 V to 0.8 V with a scanning rate of 1 V/s.

Electrical functional characterization was first performed with the microelectrode tip positioned well above the floor of the glass beaker to establish baseline device performance (i.e. measurement number 1 associated with an unloaded, un-deformed state). The harness holding the PCB was then manually lowered until the microelectrode tip came into contact with the bottom of the glass beaker, thereby imposing longitudinal uniaxial compression. The harness was then lowered further until elastic buckling was observed, at which point the harness position was fixed and EIS and CV measurements were taken again (i.e. measurement number 2 associated with loaded, elastically buckled state). The harness was then further lowered until plastic deformation of the devices was induced (as verified by permanent deformation after unloading). Afterwards, the harness was raised sufficiently to fully unload the plastically-deformed device, and further EIS and CV measurements were made (i.e. measurement number 3 associated with unloaded, plastically deformed state). Two to three samples of each length variations were tested.

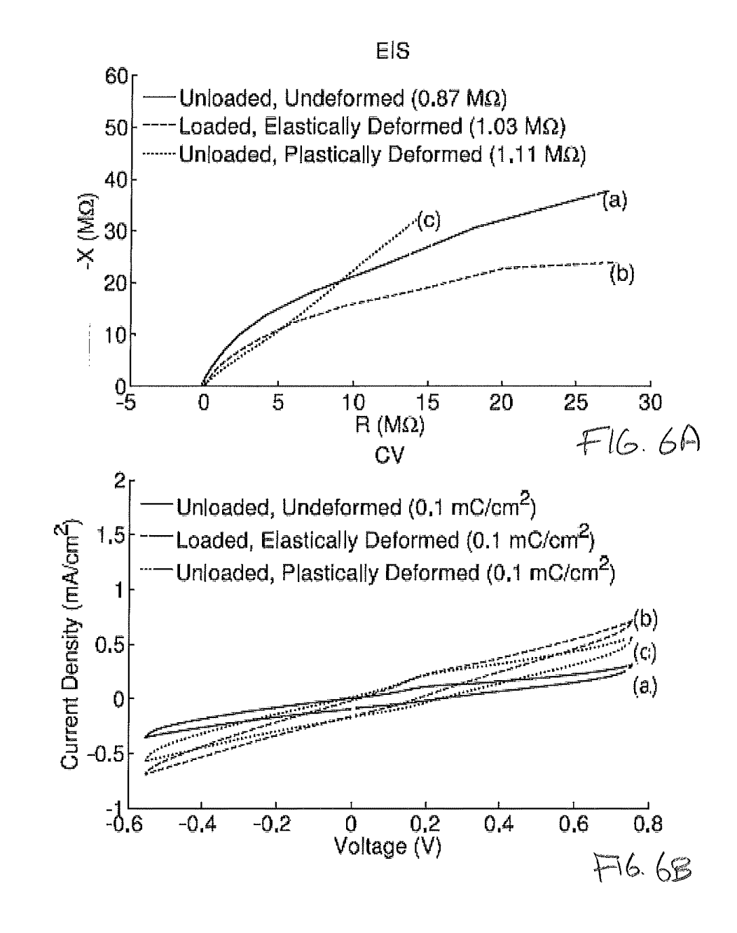

Referring to FIGS. 6A and 6B, EIS and CV results of the electrical functional characterization are provided for a single electrode site located near the middle of the tensile face of a 2 mm length Ti-based microelectrode array for the unloaded, un-deformed state (i.e. measurements made with the device suspended in the in vitro characterization apparatus prior to mechanical loading against the beaker floor), loaded, elastically deformed state, and unloaded plastically deformed state. Measured EIS results for the Ti-based microelectrode arrays in the unloaded, un-deformed state indicated impedance values between 0.20 and 1.11 MΩ at 1 kHz frequency for electrode site diameters of 40 μm, while a range of 0.8-5.13 MΩ was found for electrode site diameters of 23 μM Measured CV results indicated maximum charge carrying capacities ranging from 0.1 to 1.9 mC/cm² for all design variants.

As depicted in FIG. 6A increased impedance between the un-deformed and deformed states suggests a degradation of the electrical properties of the site Similar variations in electrical impedances were observed during testing of selected electrode sites in other devices. Moreover, while most devices were observed to buckle such that tensile stress was imposed on the dielectric stack, a similar trend in performance degradation was observed for devices in which buckling resulted in compressive stress imposed on the dielectric stack.

As mentioned above, a primary intent for the in vitro studies was to determine adequacy of Ti-based arrays based on a comparison with available Si-based arrays prior to undertaking in vivo studies. The impedance measurements for Ti-based arrays in the unloaded, un-deformed state (at a 40 μm diameter electrode site providing a range of 0.20 to 1.11 MΩ, and at a 23 μm diameter site range providing a range of 0.8 to 5.13 MΩ) were in sufficient agreement with reported values for Si-based commercial arrays (depending on the electrode site diameter providing a range of 0.5 to 3.0 MΩ, as reported in the prior art) to ensure the Ti-based arrays would provide adequate recording performance, prior to undertaking in vivo studies Similarly, measured charge carrying capacities (providing a range of 0.1 to 1.9 mC/cm²) were observed to be in fair agreement with Si-based arrays which utilize gold electrode sites of similar sizes, as reported in the prior art.

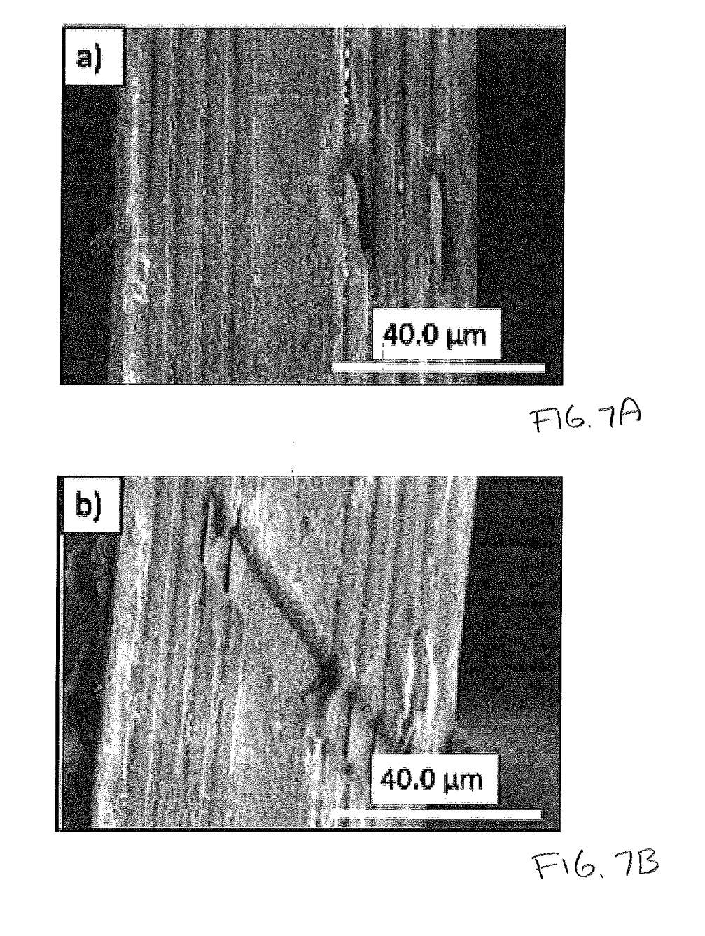

Also, as mentioned above a secondary intent of the in vitro characterization studies was to determine whether Ti-based arrays were able to maintain recording functionality after being subjected to elastic buckling or plastic deformation. Referring to FIGS. 7A and 7B, scanning electron micrographs of typical examples of dielectric damage resulting from plastic deformation of the Ti-based microelectrode arrays are provided. These figures demonstrate that T-based arrays are able to retain a significant portion of their original recording functionality during elastic buckling, as well as after plastic deformation and unloading. Contrasting this ability with the Si-based arrays in which elastic buckling typically results in fragmentation of the array, suggests that Ti-based arrays provide greater recording reliability relative to silicon arrays, since some level of recording functionality can be retained despite overloading.

### Additional Cleaning Procedure

Upon inspection, it was found that many of the electrode sites on some of the microelectrodes, particularly those with 23 μM electrode sites, still had some residue attributed to Si₃N₄ or other contaminants, such as polymeric residues resulting from the dry etching steps. These contaminations led to a significantly increase in impedance values. In order to remove some of the contamination, a cleaning step was performed using the EIS and CV testing apparatus. A 1.5 V DC voltage drop was applied across each recording site for 1 minute to generate an electrolysis effect.

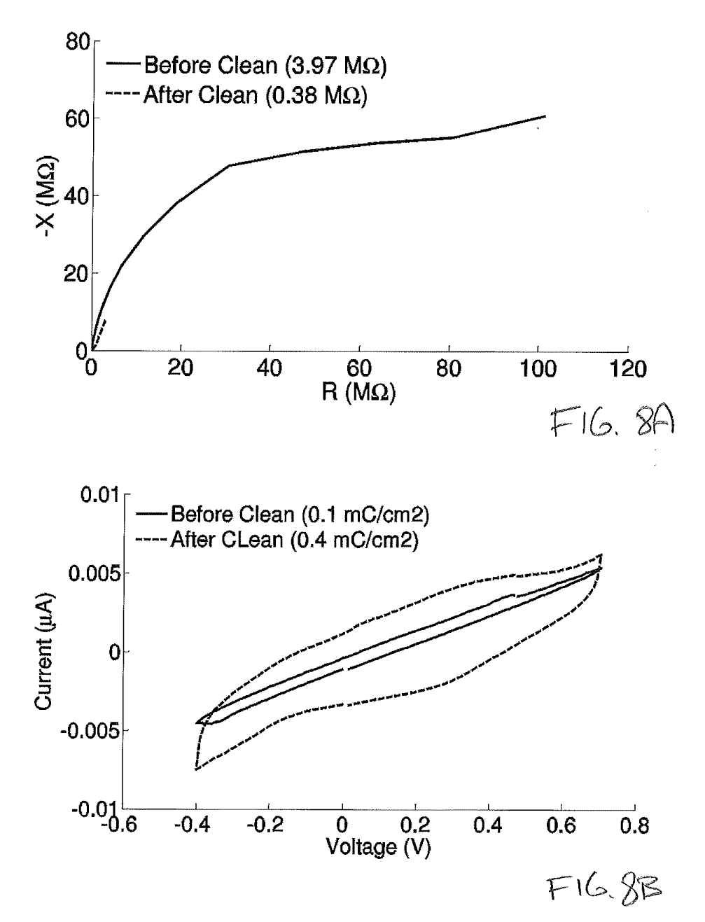

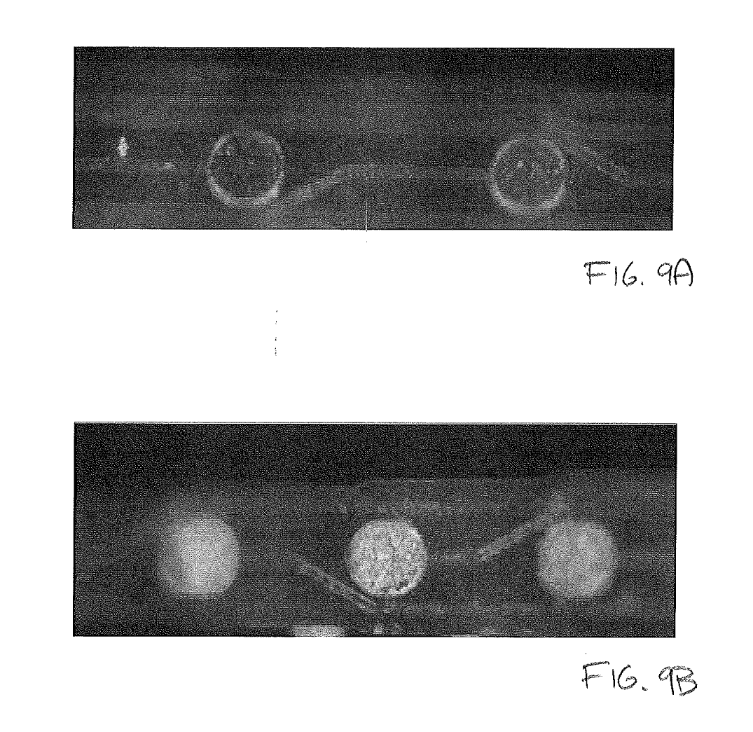

The electrolysis generates a large amount of energy which assists to remove residue on the electrode sites. The oxygen and hydrogen bubbles generated at the surface also helped to remove residue. The impedance value range of one microelectrode array with 23 μm diameter electrode sites decreased from 2.44-5.13 MΩ to 0.97-1.38 MΩ, while a microelectrode array with 40 μm diameter electrode sites had an impedance range drop from 1.26-1.98 MΩ to 0.21-0.32 MΩ. FIGS. 8A and 8B, provide graphs of the results of EIS and CV measurements for before and after the cleaning process for a single microelectrode site. FIGS. 9A and 9B depict optical micrographs of reflectivity of recording site before and after the cleaning process, where the improved reflectivity of the electrode sites indicates the removal of surface residues by the cleaning process.

### In Vivo Testing

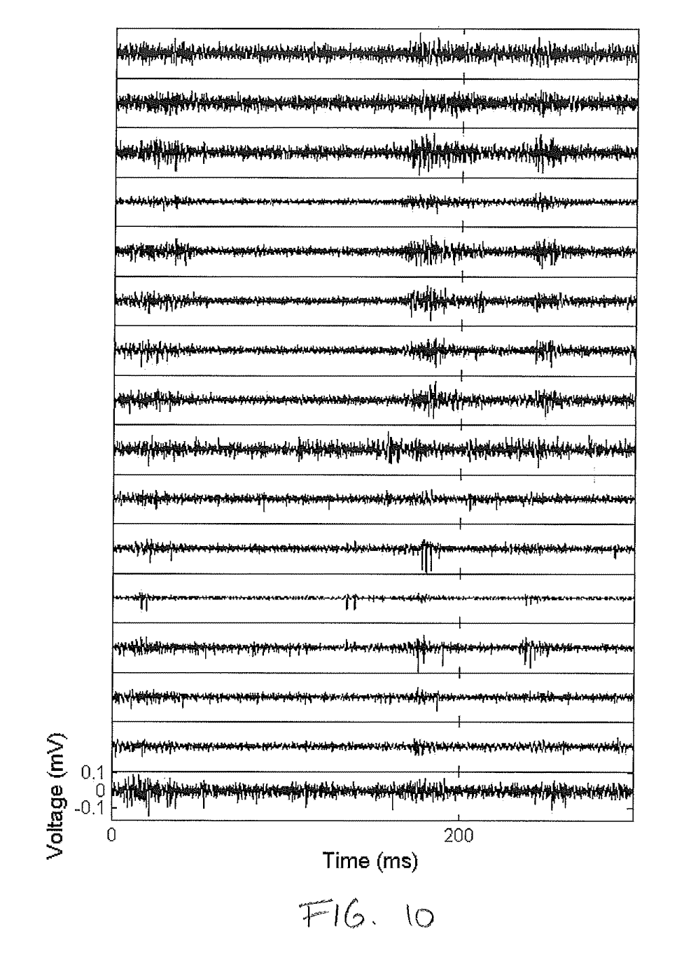

Referring to FIG. 10, an exemplary plot of in vivo electrical signals that are present at the electrode sites and are transferred to the pads via electrical traces is provided. To verify the placement and functionality of the electrodes, broadband, acoustic noise stimuli were used in order to induce multi-channel responses. The plot of FIG. 10 shows recording traces from a single microelectrode array implanted in the auditory thalamus and cortex of a rat. The top 8 sites reflect recordings from the cortex, while the bottom 8 sites reflect recordings from the thalamus. There were several easily isolatable action potentials located near the recording traces. Table 1 gives the recorded responses of the action potentials. The average signal-to-noise ratio was 5.23, which is comparable to “good” recordings from conventional microelectrode arrays, as reported in the prior art.

TABLE 1
Action potential recordings.

| | | | | Standard |
|---|---|---|---|---|
| | Min | Max | Mean | Deviation |
| Amplitude (nV) | 62.87 | 432.55 | 173.91 | 109.79 |
| Noise (nV) | 16.89 | 39.83 | 31.93 | 6.18 |
| S/N Ratio | 3.09 | 13.05 | 5.23 | 3.05 |
| Units | 0 | 2 | 0.81 | 0.75 |
| Total Units: 13 | | | | |

Initially, successful acoustic threshold physiology was observed in the 4.9 mm microelectrode array (FIG. 2), but only within the auditory cortex, thus indicating that device length was insufficient to reach desired nuclei in the thalamus. Through initial recording with a 5.4 mm microelectrode (FIG. 2), normal acoustic threshold physiology was observed on 9 of the 16 sites. Normal acoustic threshold physiology was observed on 15 of the 16 sites of the third microelectrode, which had undergone the cleaning step (discussed above) prior to in vivo use.

Those skilled in the art will recognize that numerous modifications can be made to the specific implementations described above. Therefore, the following claims are not to be limited to the specific embodiments illustrated and described above. The claims, as originally presented and as they may be amended, encompass variations, alternatives, modifications, improvements, equivalents, and substantial equivalents of the embodiments and teachings disclosed herein, including those that are presently unforeseen or unappreciated, and that, for example, may arise from applicants/patentees and others.

1. A microelectrode array comprising:
a top portion;
a plurality of pads positioned on the top portion; and
a shank portion, the shank portion including:
a titanium substrate;
a dielectric structure positioned on the titanium substrate; and
a metallization layer embedded in the dielectric structure, the metallization layer including a plurality of electrode sites distributed longitudinally along the shank portion, and a plurality of electrical traces, wherein the dielectric structure provides an access window over each of the plurality of the electrode sites and each of the plurality of electrical traces electrically connects a corresponding electrode site of the plurality of electrode sites to a corresponding pad of the plurality of pads.

2. The microelectrode array of claim 1, the shank portion further comprises:
a first portion, the first portion includes a first plurality of electrode sites and a first plurality of electrical traces, each electrical trace of the first plurality of electrical traces connects a corresponding electrode site of the first plurality of electrode sites to a corresponding pad of the plurality of pads; and
a second portion, the second portion includes a second plurality of electrode sites and a second plurality of electrical traces, each electrical trace of the second plurality of electrical traces connects a corresponding electrode site of the second plurality of electrode sites to a corresponding pad of the plurality of pads.

3. The microelectrode array of claim 1, wherein the electrode sites is one of gold, titanium nitride, iridium oxide, Polyethylenedioxythiophene (PEDOT), and carbon nanotubes.

4. The microelectrode array of claim 1, the dielectric structure includes silicon oxide (SiO₂) and silicon nitride (Si₃N₄).

5. The microelectrode array of claim 1, wherein each electrode site of the plurality of electrode sites is longitudinally separated from another electrode site by a pitch of one of about 50 μm, 75 μm, and 100 μm.

6. The microelectrode array of claim 1, wherein each electrical trace of the plurality of electrical traces includes a width of about 5 μm.

7. The microelectrode array of claim 1, wherein each electrode site of the plurality of electrode site is circular and is defined by a diameter of one of about 40 μm and 23 μm.

8. The microelectrode array of claim 1, wherein the width of the shank portion increases corresponding to longitudinal placement of each electrode site of the plurality of electrode sites.

9. The microelectrode array of claim 8, wherein the shank portion is defined by a minimum width around a first electrode site of the plurality of electrode sites of one of about 65 μm and about 48 μm to a maximum effective width of one of about 209 μm and 192 μm around a second electrode site of the plurality of electrode sites based on the diameter of the plurality of electrode sites.

10. The microelectrode array of claim 8, wherein the longitudinal length of the shank portion is about 2 mm.

11. The microelectrode array of claim 10, wherein the shank portion is defined by a buckling resistance of about 100 mN.

12. The microelectrode array of claim 2, wherein the longitudinal length of the first shank portion is about 2 mm and the longitudinal length of the second shank portion is one of about 2.9 and 3.4 mm.

13. The microelectrode array of claim 12, wherein the shank portion is defined by a buckling resistance of about 20 mN associated with the second shank portion having a longitudinal length of about 2.9 mm and a buckling resistance of about 19.41 mN associated with the second shank portion having a longitudinal length of about 3.4 mm.

14. The microelectrode array of claim 1, each electrode site of the plurality of electrode sites is substantially free of remnants of the dielectric structure, where the plurality electrode sites are cleaned by an electrolysis process to remove the dielectric structure.

15. The microelectrode array of claim 2, each electrode site of the first and second pluralities of electrode sites is substantially free of the dielectric structure, where the first and second pluralities of electrode sites are cleaned by an electrolysis process to remove remnants of the dielectric structure.

16. A microelectrode array comprising:
a top portion;
a plurality of pads positioned on the top portion; and
a shank portion, the shank portion including:
a titanium substrate,
a dielectric structure positioned on the titanium substrate,
a metallization layer embedded in the dielectric structure, the metallization layer defining:
a first plurality of electrode sites and a first plurality of electrical traces positioned on a first shank portion, each electrical trace of the first plurality of electrical traces connects a corresponding electrode site of the first plurality of electrode sites to a corresponding pad of the plurality of pads, and
a second plurality of electrode sites and a second plurality of electrical traces positioned on a second shank portion, each electrical trace of the second plurality of electrical traces connects a corresponding electrode site of the second plurality of electrode sites to a corresponding pad of the plurality of pads,
wherein each electrode site of the first and second pluralities of electrode sites is substantially free of the dielectric structure, where the first and second pluralities of electrode sites are cleaned by an electrolysis process to remove remnants of the dielectric structure.
